(19) United States
(12) Patent Application Publication
(10) Pub. No.: US 2011/0262484 A1
(43) Pub. Date: Oct. 27, 2011

(54) OUTER MEMBRANE VESICLE PRIME-PROTEIN BOOST VACCINE

(75) Inventor: Ian Feavers, London (GB)

(73) Assignee: Health Protection Agency, London (GB)

(21) Appl. No.: 13/062,168

(22) PCT Filed: Sep. 4, 2009
(86) PCT No.: PCT/GB2009/002129

§ 371 Date: Jul. 11, 2011

(30) Foreign Application Priority Data
Sep. 5, 2008 (GB) 0816284.4

(51) Int. Cl. A61K 39/095; A61P 37/04; A61P 31/04; A61P 19/02

(52) U.S. Cl. 424/250.1

(57) ABSTRACT

The present invention provides a method of immunizing a subject against a disease caused by Neisseria meningitidis, comprising administering to the subject a primer composition comprising a meningococcal outer membrane vesicle preparation, and a booster composition comprising a meningococcal protein antigen preparation. Vaccine combinations comprising primer and booster compositions and associated uses are also provided.

### FIELD

The present invention relates to the field of methods for promoting an immune response against meningococcal pathogens. In particular, the invention relates to a method for immunising a subject against a disease caused by Neisseria meningitidis, as well as to a combination for prime-boost vaccination against a disease such as bacterial meningitis.

### BACKGROUND

Neisseria meningitidis (also known as meningococcus) is a non-motile, Gram-negative capsulate bacterium that is the most common cause of bacterial meningitis and septicaemia. It colonises the pharynx, causing meningitis and, occasionally, septicaemia in the absence of meningitis. In the United States the attack rate is 0.6-1 per 100,000 persons per year, and it can be much greater during outbreaks (see Lieberman et al. (1996) JAMA 275 (19): 1499-1503; Schuchat et al (1997) N Engl J Med 337 (14): 970-976). In Europe attack rates vary considerably between countries, ranging from 0.3-9 cases per 100,000. In developing countries, endemic disease rates are much higher and during epidemics incidence rates can reach 500 cases per 100,000 persons per year. Mortality is extremely high, at 5-10% across Europe, 10-20% in the United States, and much higher in developing countries. N. meningitidis is also associated with other severe infections including meningococcal arthritis and rarely pneumonia.

Risk groups include infants and young children, refugees, household contacts of patients, military recruits, college students and microbiologists who work with live isolates. Besides the high case fatality rates, survivors may have serious long-term effects such as brain damage, hearing loss, learning disability and limb amputation.

Twelve serogroups of N. meningitidis have been identified and four (A, B, C and W135) are the major pathogens. The serogroup is determined by the immunochemistry of the meningococcal capsular polysaccharide. Groups B and C are the commonest types causing meningitis in Europe and the Americas. Group Y is associated with pneumonia and incidence is currently increasing in the US. Following the introduction of the conjugate vaccine against Haemophilus influenzae, N. meningitidis is the major cause of bacterial meningitis at all ages in the United States.

Licensed meningococcal vaccines are based on the capsular polysaccharide. Tetravalent polysaccharide preparations offer protection against groups A, C, Y and W135 but are not effective in young children, the age-group most susceptible to meningococcal disease. Conjugate vaccines, in which the polysaccharide is chemical linked to a carrier protein, are more effective in children and have the potential to offer herd immunity in an immunised population. Conjugate vaccines that target group C disease only are widely used in Europe and a tetravalent conjugate (A, C, W135 and Y) is licensed in the USA. The group B meningococcal capsular polysaccharide is not used as a vaccine antigen. Serogroup B of N. meningitidis is currently responsible for approximately 50% of total meningitis in the United States, Europe, and South America. The polysaccharide approach cannot be used because the menB capsular polysaccharide is a polymer of α(2-8)linked N-acetyl neuraminic acid that is also present in mammalian tissue. This results in tolerance to the antigen; indeed, if a response were elicited, there is concern it might be anti-self, and therefore undesirable.

One of the various approaches to immunising against infection by Neisseria meningitidis (meningococcus) is to use outer membrane vesicles (OMVs). An efficacious outer-membrane vesicle (OMV) vaccine against serogroup B has been produced by the Norwegian National Institute of Public Health [see Bjune et al. (1991) Lancet 338 (8775): 1093-96]. Whilst this vaccine is safe and prevents group B meningococcal (NmB) disease, its efficacy is limited to the strain used to make the vaccine. This restricted efficacy is explained by the antigenic variability of the immunodominant PorA antigen, which is also historically referred to as the Class 1 outer membrane protein (OMP). Other vaccines based around outer-membrane preparations have also been reported.

The ‘RIVM’ or Netherlands Vaccine Institute (NVI) vaccine is based on OMVs containing six different PorA subtypes. It has been shown to be immunogenic in children in phase II clinical trials (de Kleijn et al (2001) Vaccine 20:352-358).

U.S. Pat. Nos. 5,597,572 & 5,747,653 disclose a vaccine against different pathogenic serotypes of serogroup B meningococcus based on OMVs which retain a protein complex of 65-kDa. WO90/06696 discloses a vaccine comprising OMVs from genetically-engineered meningococcal strains, with the OMVs comprising: at least one Class 1 OMP but not comprising a Class 2/3 OMP.

WO94/08021 discloses OMVs comprising OMPs which have mutations in their surface loops and OMVs comprising derivatives of meningococcal lipopolysaccharide (LPS). WO01/91788 discloses a process for preparing OMV-based vaccines for serogroup A meningococcus.

There have been various proposals to improve OMV efficacy. WO00/25811 discloses compositions comprising OMVs supplemented with transferrin binding proteins (e.g. TbpA and TbpB) and/or Cu,Zn-superoxide dismutase. WO01/52885 discloses compositions comprising OMVs supplemented by various proteins.

The failure of meningococcal OMVs to elicit cross-protection against non-homologous serosubtypes limits their use as general vaccines, but they can be very useful in epidemic situations where disease is characterised by pathogenic strains that are essentially clonal. Thus the Finlay Institute vaccine (VA-MENGOC-BC™) has been useful in Latin America, where serogroup B disease had been dominated by the P1.19,15 serotype, but has not been effective elsewhere (Sacchi et al. (1998) Rev Inst Med Trop Sao Paulo 40:65-70). Similarly, the Chiron MeNZB™ vaccine has been targeted at the epidemic strain (P1.7b,4, known as P1.7-2,4 by recent nomenclature) that has been prevalent in New Zealand since 1991.

Despite the success of such vaccines, some individuals show a deficient response to immunization such that an effective level of immunity is not induced. This is particularly common amongst infants, who tend to respond poorly to immunization with OMVs. Since the very young are most at risk of meningococcal disease, this is a major problem with regard to use of OMV-based vaccines.

Thus there is a need for new and improved vaccination strategies for immunizing against meningococcal disease, for instance for enhancing immune responses against OMVs and associated protein antigens.

### SUMMARY OF THE INVENTION

In one aspect the present invention provides a method of promoting an immune response in a subject against a meningococcus bacterium, comprising administering to the subject a primer composition comprising a meningococcal OMV preparation, and a booster composition comprising a meningococcal protein antigen preparation.

In another aspect, the present invention provides a method of immunizing a subject against a disease caused by Neisseria meningitidis, comprising administering to the subject a primer composition comprising an OMV preparation, and a booster composition comprising a protein antigen preparation.

In another aspect, the present invention provides a pharmaceutical combination comprising a primer composition comprising a meningococcal OMV preparation, and a booster composition comprising a meningococcal protein antigen preparation.

In another aspect, the present invention provides a combined vaccine, comprising a primer composition comprising a meningococcal outer membrane vesicle preparation, and a booster composition comprising a meningococcal protein antigen preparation, for separate or subsequent administration for the prevention of meningococcal infection.

In another aspect, the present invention provides use of a primer composition comprising a meningococcal outer membrane vesicle preparation, and a booster composition comprising a meningococcal protein antigen preparation, for the preparation of a combined vaccine for prevention or treatment of a meningococcal infection.

The method may comprise promoting, including inducing or augmenting, an immune response against a meningococcus bacterium or an antigen derived therefrom. For instance the method may involve immunizing or vaccinating a subject against N. meningitidis, e.g. for preventing or treating meningococcal disease. The disease to be prevented or treated may be, for example, bacterial (meningococcal) meningitis, septicaemia, meningococcal arthritis or pneumonia. Preferably the disease is meningitis or septicaemia.

The meningococcal serogroup or strain to be prevented or treated, e.g. against which the immunization provides protection, may be any pathogenic serotype, particularly A, B, C, Y or W135. The outer membrane vesicles (OMV) and outer membrane protein (OMP) may accordingly be derived from such a serotype, e.g. from serotype A, B, C, Y or W135. Preferably the method involves immunizing or promoting an immune response against N. meningitidis serogroup B.

Preferably the meninogococcal protein antigen preparation comprises a meningococcal outer membrane protein preparation. The protein antigen or outer membrane protein preparation is preferably a purified protein preparation. The OMP may be, for example, one or more of the following proteins or an immunogenic fragment thereof: PorA, PorB, FetA, NadA, transferrin binding proteins (e.g. TbpA and TbpB), lactoferrin binding proteins, haem and haptoglobin receptors, complement factor H binding protein, Opa protein, NspA, Omp85, PilQ and Cu, Zn-superoxide dismutase. Preferably the OMP is PorA.

In one embodiment, the primer composition and/or the booster composition comprises an adjuvant. Preferably the adjuvant comprises monophosphoryl lipid A or aluminium hydroxide.

In one embodiment the OMV preparation does not comprise a purified meningococcal protein antigen or OMP, e.g. the OMV preparation may consist essentially of outer membrane vesicles. Likewise, in particular embodiments the primer composition does not comprise a purified meningococcal protein antigen or OMP, e.g. the primer composition may consist essentially of OMVs and one or more pharmaceutically acceptable excipients, carriers and/or adjuvants.

In one embodiment the meningococcal protein antigen (e.g. OMP) preparation does not comprise outer membrane vesicles, e.g. the meningococcal protein antigen or OMP preparation consists essentially of one or more purified meningococcal protein antigens (e.g. OMPs). Likewise, in particular embodiments the booster composition does not comprise OMVs, e.g. the the booster composition may consist essentially of one or more purified meningococcal protein antigens (e.g. OMPs) and one or more pharmaceutically acceptable excipients, carriers and/or adjuvants.

The booster composition may be administered to the subject at various times after the primer composition has been administered. For example, in one embodiment the booster composition is administered 2 to 10 weeks, preferably 3 to 6 weeks after the primer composition.

Embodiments of the present invention may advantageously promote immune responses to meningococcal antigens, thereby enhancing the effectiveness of vaccines against diseases such as meningitis.

### BRIEF DESCRIPTION OF THE FIGURE

FIG. 1 shows serum bactericidal antibody titres for groups of mice primed and then boosted with different permutations of OMVs and purified PorA protein. The experiment was repeated with Al(OH)₃ and MPL as adjuvants. Group 1: OMV prime, OMV boost. Group 2: OMV prime, PorA protein boost. Group 3: PorA protein prime, OMV boost. Group 4: PorA protein prime, PorA protein boost.

### DETAILED DESCRIPTION OF THE INVENTION

### Outer Membrane Vesicles (OMVs)

In embodiments of the present invention, the primer composition comprises an outer membrane vesicle (OMV) preparation derived from Neisseria meningitidis. The OMV preparation may be any form of composition comprising meningococcal OMVs. Thus ‘OMV’ includes any proteoliposomic vesicle obtained by disrupting a bacterial outer membrane to form vesicles of the outer membrane that include protein components of the outer membrane.

OMVs may be prepared artificially from bacteria e.g. by detergent treatment, or by non-detergent means, for instance as described in WO2004/019977. The term also encompasses blebs, microvesicles (e.g. MVs as described in WO02/09643) and ‘native OMVs’ (e.g. ‘NOMVs’ as described in Katial et al. (2002) Infect. Immun. 70:702-707), which are naturally-occurring membrane vesicles that form spontaneously during bacterial growth and are released into culture medium.

MVs can be obtained by culturing Neisseria in broth culture medium, separating whole cells from the smaller MVs in the broth culture medium (e.g. by filtration or by low-speed centrifugation to pellet only the cells and not the smaller vesicles), and then collecting the MVs from the cell-depleted medium (e.g. by filtration, by differential precipitation or aggregation of MVs, by high-speed centrifugation to pellet the MVs). Stains for use in production of MVs can generally be selected on the basis of the amount of MVs produced in culture e.g. U.S. Pat. No. 6,180,111 and WO01/34642 describe Neisseria with high MV production.

OMVs can be prepared in various ways. Methods for obtaining suitable preparations are disclosed in, for instance, the references cited herein. Techniques for forming OMVs include treating bacteria with a bile acid salt detergent e.g. salts of lithocholic acid, chenodeoxycholic acid, ursodeoxycholic acid, deoxycholic acid, cholic acid, ursocholic acid, etc., with sodium deoxycholate being preferred for treating Neisseria at a pH sufficiently high not to precipitate the detergent (see EP0011243 and WO01/91788). Other techniques may be performed substantially in the absence of detergent using techniques such as sonication, homogenisation, microfluidisation, cavitation, osmotic shock, grinding, French press, blending, etc (see WO2004/019977).

A preferred method for OMV preparation involves ultrafiltration instead of high speed centrifugation on crude OMVs (see WO2005/004908). This allows much larger amounts of OMV-containing supernatant to be processed in a much shorter time (typically>15 litres in 4 hours, compared to<1.5 litres in 10 hours), and avoids the need to redisperse OMVs after centrifugation. Ultracentrifugation allows large quantities of OMVs to be prepared much more easily, and permits the rapid production of OMVs from a stain of choice, for use in vaccine preparation.

In specific embodiments, the primer composition may be a known vaccine against Neisseria meningitidis comprising OMVs, e.g. RIVM, VA-MENGOC-BC, MeNZB or MenBvac. The MenBvac™ product comprises OMVs prepared from Norwegian strain H44/76. OMVs prepared from New Zealand strain NZ98/254 form the basis of the MeNZB™ product. Their safety and efficacy have been confirmed. Both MeNZB™ and MenBvac™ include OMVs at a concentration of 50 μg/ml (measured as amount of protein) in a 0.5 ml dose.

Meningococcal Strains used for Vaccine Preparation

Identifying the serosubtype of a meningococcal strain of interest can be achieved using standard techniques, based on the class 1 porin OMP (PorA). Meningococcal strains used according to the invention include strains that express multiple serosubtypes, i.e. multiple PorA alleles. In other embodiments the meningococcal strain expresses a single PorA sequence i.e. is of a single serosubtype.

Meningococcal strains used according to the invention will generally be in, but are not limited to, one of the following serogroups: A, B, C, W135, or Y. In alternative embodiments, strains from any other meningococcal serogroup may be used in the invention, including for example serogroup X. Meningococci used according to the invention may be of any serotype e.g. 1, 2a, 2b, 4, 14, 15, 16, etc.) and/or of any immunotype e.g. L1; L3,3,7; L10; etc.). The meningococci may be from any suitable lineage, including hyperinvasive and hypervirulent lineages e.g. any of the following seven hypervirulent lineages: subgroup I; subgroup III; subgroup IV-I; ET-5 complex; ET-37 complex; A4 cluster; lineage 3. These lineages have been defined by multilocus enzyme electrophoresis (MLEE), but multilocus sequence typing (MLST) has also been used to classify meningococci (see Maiden et al (1998) PNAS USA 95:3140-3145) e.g. the ET-37 complex is the ST-11 complex by MLST, the ET-5 complex is ST-32 (ET-5), lineage 3 is ST-41/44, etc.

Meningococci may have one or more knockout mutations of gene(s). To reduce pyrogenic activity, for instance, the bacterium should have low endotoxin (LPS) levels, and this can be achieved by knockout of enzymes involved in LPS biosynthesis. Suitable mutant bacteria are already known e.g. mutant Neisseria (see WO99/10497). Processes for preparing LPS-depleted outer membranes from Gram-negative bacteria are disclosed in EP0624376.

As well as down-regulating expression of specific proteins, the bacterium may over-express (relative to the corresponding wild-type strain) immunogens such as NspA, protein 287, protein 741, TbpA, TbpB, superoxide dismutase, etc. The bacterium may also express one or more genes that are not endogenous. For example, the invention may use a recombinant strain that expresses new genes relative to the corresponding wild-type strain. Expression of non-endogenous genes in this way can be achieved by various techniques e.g. chromosomal insertion (as used for introducing multiple PorA genes as described in van der Ley et al. (1995) Vaccine 13:401-7), knockin mutations, expression from extra-chromosomal vectors e.g. from plasmids), etc.

The meningococcus is preferably in serogroup B. In specific embodiments, OMVs may be prepared from a meningococcus having one of the following serosubtypes: P1.2; P1.2,5; P1.4; P1.5; P1.5,2; P1.5,c; P1.5c,10; P1.7-2.4, P1.7,16; P1.7,16b; P1.7h,4; P1.9; P1.15; P1.9,15; P1.12,13; P1.13; P1.14; P1.21,16; P1.22,14. However, any suitable meningococcal serosubtype may be used in the present invention, including for example any of those mentioned at http://neisseria.org/, see for example http://neisseria.org/perl/agdbnet/agdbnet.pl?file=poravr.xml.

### OMV Dosing

Existing meningococcal OMV vaccines offer pharmaceutical, posological and formulation guidance for preparing the primer composition in performing the invention. For example, VA-MENGOC-BC™ is an injectable suspension in 0.5 ml that contains 50 μg OMV from strain Cu-385-83 and 50 μg serogroup C capsular polysaccharide, absorbed to 2 mg of an aluminium hydroxide gel, plus 0.01% thiomersal and phosphate buffer. MeNZB™ is also a 0.5 ml suspension, and contains 25 μg OMV from strain NZ98/254 adsorbed on 1.65 mg of an aluminium hydroxide adjuvant, with a histidine buffer and sodium chloride. MenBvac is similar to MeNZB™, but is prepared from strain 44/76.

The concentration of OMVs for use in the primer composition may be selected such that it provides protective immunity, after administration of the booster composition to a subject. The concentration of OMVs in primer compositions of the invention will generally be between 10 and 500 μg/ml, preferably between 25 and 200 μg/ml, and more preferably about 50 μg/ml or about 100 μg/ml (e.g. expressed in terms of total protein in the OMVs).

In certain embodiments, the primer composition may include OMVs from more than one meningococcal serosubtype. In this case doses for individual serosubtypes may be reduced without loss of efficacy.

Meningococcal Protein Antigen (e.g. OMP) Preparations

According to the present invention, the booster composition may comprise one or more specific meningococcal protein antigens (preferably from serogroup B), or immunogenic fragments thereof. By “meningococcal protein antigen” it is intended to encompass any protein, polypeptide or fragment thereof which is derived from N. meningitidis and which is capable of generating an immune response when introduced into a subject (e.g. a human). Preferably the meningococcal protein antigen is specific to N. meningitidis.

Preferably the meningococcal protein antigen is an outer membrane protein. By “outer membrane protein” it is intended to encompass any protein found on the outer surface of N. meningitidis, e.g. any protein which can be detected as being located in or associated with the outer membrane of meningococcus. For example, an outer membrane protein antigen such as disclosed in WO2004/048404, WO99/24578, WO99/36544, WO99/57280, WO00/22430, WO96/29412, WO01/64920, WO03/020756 or WO2004/032958 may be used. Suitable OM proteins include, but are not limited to, PorA, PorB, FetA, NadA, transferrin binding proteins (e.g. TbpA and TbpB), lactoferrin binding proteins, haem and haptoglobin receptors, complement factor H binding protein, Opa protein, NspA, Omp85, PilQ and Cu, Zn-superoxide dismutase, including immunogenic fragments thereof.

The booster composition or OMP preparation may comprise a single protein antigen, or small number of defined antigens may be added (a mixture of 10 or fewer (e.g. 9, 8, 7, 6, 5, 4, 3, 2) purified antigens). Immunogenic fragments may comprise at least n consecutive amino acids from said sequences, wherein n is 7 or more (eg. 8, 10, 12, 14, 16, 18, 20, 25, 30, 35, 40, 50, 60, 70, 80, 90, 100, 150, 200, 250 or more). Preferred fragments comprise an epitope from the relevant sequence. More than one (e.g. 2, 3, 4, 5, 6) of these polypeptides may be included.

The meningococcal protein antigen preparation is preferably a purified protein preparation. Suitable meningococcal proteins, including outer membrane proteins, for use in the invention may be purified from appropriate meningococcal strains or produced e.g. in recombinant cells, for instance as described in the above publications. The polypeptide and DNA sequences of many OMPs are known and are available from publicly-accessible databases.

Pharmaceutical Compositions containing OMVs or Meningococcal Protein Antigens (e.g. OMPs)

The primer and/or booster compositions of the invention may be pharmaceutical compositions that include a pharmaceutically acceptable carrier. Such compositions can be prepared using a process comprising the step of admixing OMVs or meningococcal protein antigens (e.g. OMPs) with the pharmaceutically acceptable carrier. Typical ‘pharmaceutically acceptable carriers’ include any carrier that does not itself induce the production of antibodies harmful to the individual receiving the composition.

Suitable carriers are typically large, slowly metabolised macromolecules such as proteins, polysaccharides, polylactic acids, polyglycolic acids, polymeric amino acids, amino acid copolymers, and lipid aggregates (such as oil droplets or liposomes). Such carriers are well known to those of ordinary skill in the art. The vaccines may also contain diluents, such as water, saline, glycerol, etc. Additionally, auxiliary substances, such as wetting or emulsifying agents, pH buffering substances, sucrose, and the like, may be present. Sterile pyrogen-free, phosphate-buffered physiologic saline is a typical carrier e.g. based on water for injection). A thorough discussion of pharmaceutically acceptable excipients is available in Gennaro (2000) Remington: The Science and Practice of Pharmacy. 20th edition, ISBN: 0683306472.

Primer and/or booster compositions of the invention will typically be in aqueous form (e.g. solutions or suspensions) rather than in a dried form (e.g. lyophilised). Aqueous compositions are also suitable for reconstituting vaccines from a lyophilised form. Where a composition of the invention is to be used for such extemporaneous reconstitution, the invention provides a kit, which may comprise two or more vials, or may comprise one or more ready-filled syringes and one or more vials, with the aqueous contents of the syringe being used to reactivate the dried contents of the vial prior to injection.

Primer and/or booster compositions of the invention may be presented in vials, or they may be presented in ready-filled syringes. The syringes may be supplied with or without needles. Compositions may be packaged in unit dose form or in multiple dose form. A syringe will generally include a single dose of the composition, whereas a vial may include a single dose or multiple doses. For multiple dose forms, therefore, vials are preferred to pre-filled syringes.

Effective dosage volumes can be routinely established, but a typical human dose of the composition has a volume of about 0.5 ml e.g. for intramuscular injection. The RIVM OMV-based vaccine was administered in a 0.5 ml volume by intramuscular injection to the thigh or upper arm. MeNZB™ is administered in a 0.5 ml by intramuscular injection to the anterolateral thigh or the deltoid region of the arm. Similar doses may be used for other delivery routes e.g. an intranasal OMV-based vaccine for atomisation may have a volume of about 100 μl or about 130 μl per spray, with four sprays administered to give a total dose of about 0.5 ml.

The pH of the composition is preferably between 6 and 8, and more preferably between 6.5 and 7.5 (e.g. about 7). The pH of the RIVM OMV-based vaccine is 7.4, and a pH<7.5 is preferred for compositions of the invention. Stable pH may be maintained by the use of a buffer e.g. a Tris buffer, a phosphate buffer, or a histidine buffer. Compositions of the invention will generally include a buffer. If a composition comprises an aluminium hydroxide salt, it is preferred to use a histidine buffer e.g. at between 1-10 mM, preferably about 5 mM. The RIVM OMV-based vaccine maintains pH by using a 10 mM Tris/HCl buffer. The composition may be sterile and/or pyrogen-free. Compositions of the invention may be isotonic with respect to humans.

The primer and/or booster compositions of the invention are immunogenic, and are more preferably vaccine compositions. Vaccines according to the invention may either be prophylactic (i.e. to prevent infection) or therapeutic (i.e. to treat infection), but will typically be prophylactic. Immunogenic compositions used as vaccines comprise an immunologically effective amount of antigen(s), as well as any other components, as needed. By ‘immunologically effective amount’, it is meant that the administration of that amount to an individual, as part of a combined primer/booster regime as described herein, is effective for treatment or prevention.

This amount varies depending upon the health and physical condition of the individual to be treated, age, the taxonomic group of individual to be treated (e.g. non-human primate, primate, etc.), the capacity of the individual's immune system to synthesise antibodies, the degree of protection desired, the formulation of the vaccine, the treating doctor's assessment of the medical situation, and other relevant factors. It is expected that the amount will fall in a relatively broad range that can be determined through routine trials. The antigen content of compositions of the invention will generally be expressed in terms of the amount of protein per dose. A dose of about 0.9 mg protein per ml is typical for OMV-based intranasal vaccines.

Meningococci affect various areas of the body and so the compositions of the invention may be prepared in various forms. For example, the compositions may be prepared as injectables, either as liquid solutions or suspensions. The composition may be prepared for pulmonary administration e.g. as an inhaler, using a fine powder or a spray. The composition may be prepared as a suppository or pessary. The composition may be prepared for nasal, aural or ocular administration e.g. as spray, drops, gel or powder (see e.g. Almeida & Alpar (1996) J. Drug Targeting 3:455-467). Injectables for intramuscular administration are typical.

Primer and/or booster compositions of the invention may include an antimicrobial, particularly when packaged in multiple dose format. Antimicrobials such as thiomersal and 2-phenoxyethanol are commonly found in vaccines, but it is preferred to use either a mercury-free preservative or no preservative at all. Compositions of the invention may comprise detergent e.g. a Tween (polysorbate), such as Tween 80. Detergents are generally present at low levels e.g. <0.01%. In one embodiment, the meningococcal protein antigen preparation comprises a detergent. Detergents may assist in ensuring that the meningococcal protein (e.g. OMP) adopts an appropriate conformation for eliciting an immune response.

Primer and/or booster compositions of the invention may include residual detergent (e.g. deoxycholate) from OMV preparation. The amount of residual detergent is preferably less than 0.4 μg (more preferably less than 0.2 μg) for every μg of protein. Compositions of the invention may include LPS from meningococcus. The amount of LPS is preferably less than 0.12 μg (more preferably less than 0.05 μg) for every μg of protein. Compositions of the invention may include sodium salts (e.g. sodium chloride) to give tonicity. A concentration of 10 mg/ml NaCl is typical. The concentration of sodium chloride is preferably about 9 mg/ml.

Primer and/or booster compositions of the invention will generally be administered in conjunction with other immunoregulatory agents. In particular, compositions will usually include one or more adjuvants, and the invention provides a process for preparing a composition of the invention, comprising the step of separately admixing OMVs and/or OMPs with an adjuvant e.g. in a pharmaceutically acceptable carrier. Suitable adjuvants are discussed in Vaccine Design (1995) eds. Powell & Newman. ISBN: 030644867X, Plenum, and include, but are not limited to:

### A. Mineral-Containing Compositions

Mineral containing compositions suitable for use as adjuvants in the invention include mineral salts, such as aluminium salts and calcium salts. The invention includes mineral salts such as hydroxides (e.g. oxyhydroxides), phosphates (e.g. hydroxyphosphates, orthophosphates), sulphates, etc., or mixtures of different mineral compounds, with the compounds taking any suitable form (e.g. gel, crystalline, amorphous, etc.), and with adsorption being preferred. The mineral containing compositions may also be formulated as a particle of metal salt.

For instance the RIVM vaccine may be absorbed to either an aluminium phosphate or an aluminium hydroxide adjuvant. The MeNZB™, MenBvac™ and VA-MENINGOC-BC™ products all include an aluminium hydroxide adjuvant.

A typical dose of aluminium adjuvant is about 3.3 mg/ml (expressed as concentration of the aluminium ion).

### B. Oil Emulsions

Oil emulsion compositions suitable for use as adjuvants in the invention include squalene-water emulsions, such as MF59 (5% Squalene, 0.5% Tween 80, and 0.5% Span 85, formulated into submicron particles using a microfluidizer). Complete Freund's adjuvant (CFA) and incomplete Freund's adjuvant (IFA) may also be used when immunizing some animal species.

### C. Saponin Formulations

Saponin formulations may also be used as adjuvants in the invention. Saponins are a heterologous group of sterol glycosides and triterpenoid glycosides that are found in the bark, leaves, stems, roots and even flowers of a wide range of plant species.

Saponin from the bark of the Quillaia saponaria Molina tree have been widely studied as adjuvants. Saponin can also be commercially obtained from Smilax omata (sarsaprilla), Gypsophilla paniculata (brides veil), and Saponaria officianalis (soap root). Saponin adjuvant formulations include purified formulations, such as QS21, as well as lipid formulations, such as ISCOMs. QS21 is marketed as Stimulon™.

Saponin compositions have been purified using HPLC and RP-HPLC. Specific purified fractions using these techniques have been identified, including QS7, QS 17, QS 18, QS21, QH-A, QH-B and QH-C. Preferably, the saponin is QS21.

Saponin formulations may also comprise a sterol, such as cholesterol. Combinations of saponins and cholesterols can be used to form unique particles called immunostimulating complexes (ISCOMs). ISCOMs typically also include a phospholipid such as phosphatidylethanolamine or phosphatidylcholine. Any known saponin can be used in ISCOMs. Preferably, the ISCOM includes one or more of QuilA, QHA and QHC. Optionally, the ISCOMS may be devoid of extra detergent. Saponin based adjuvants are further discussed in e.g. Barr et al. (1998) Advanced Drug Delivery Reviews 32:247-271.

### D. Virosomes and Virus-Like Particles

Virosomes and virus-like particles (VLPs) can also be used as adjuvants in the invention. These structures generally contain one or more proteins from a virus optionally combined or formulated with a phospholipid. They are generally non-pathogenic, non-replicating and generally do not contain any of the native viral genome. The viral proteins may be recombinantly produced or isolated from whole viruses. These viral proteins suitable for use in virosomes or VLPs include proteins derived from influenza virus (such as HA or NA), Hepatitis B virus (such as core or capsid proteins), Hepatitis E virus, measles virus, Sindbis virus, Rotavirus, Foot-and-Mouth Disease virus, Retrovirus, Norwalk virus, human Papilloma virus, HIV, RNA-phages, Q[beta]-phage (such as coat proteins), GA-phage, fr-phage, AP205 phage, and Ty (such as retrotransposon Ty protein pi). VLPs and virosomes are discussed further in, for example, WO03/024481 and Gluck et al. (2002) Vaccine 20:B10-B16.

### E. Bacterial or Microbial Derivatives

Adjuvants suitable for use in the invention include bacterial or microbial derivatives such as non-toxic derivatives of enterobacterial lipopolysaccharide (LPS), Lipid A derivatives, immunostimulatory oligonucleotides and ADP-ribosylating toxins and detoxified derivatives thereof.

Non-toxic derivatives of LPS include monophosphoryl lipid A (MPL) and 3-O-deacylated MPL (3dMPL). 3dMPL is a mixture of 3 de-O-acylated monophosphoryl lipid A with 4, 5 or 6 acylated chains. A preferred ‘small particle’ form of 3 De-O-acylated monophosphoryl lipid A is disclosed in EP0689454. Such ‘small particles’ of 3dMPL are small enough to be sterile filtered through a 0.22 μm membrane. Other non-toxic LPS derivatives include monophosphoryl lipid A mimics, such as aminoalkyl glucosaminide phosphate derivatives e.g. RC-529 (Evans et al. (2003) Expert Rev Vaccines 2:219-229). Lipid A derivatives include derivatives of lipid A from Escherichia coli such as OM-174. OM-174 is described for example in Meraldi et al. (2003) Vaccine 21:2485-2491.

Immunostimulatory oligonucleotides suitable for use as adjuvants in the invention include nucleotide sequences containing a CpG motif (a dinucleotide sequence containing an unmethylated cytosine linked by a phosphate bond to a guanosine). Double-stranded RNAs and oligonucleotides containing palindromic or poly(dG) sequences have also been shown to be immunostimulatory. The CpGs can include nucleotide modifications/analogs such as phosphorothioate modifications and can be double-stranded or single-stranded. The adjuvant effect of CpG oligonucleotides is further discussed in Krieg (2003) Nature Medicine 9:831-835.

Bacterial ADP-ribosylating toxins and detoxified derivatives thereof may be used as adjuvants in the invention.

Preferably, the protein is derived from E. coli (E. coli heat labile enterotoxin ‘LT’), cholera (‘CT’), or pertussis (‘PT’). The use of detoxified ADP-ribosylating toxins as mucosal adjuvants and as parenteral adjuvants is described in WO95/17211 and WO98/42375.

### F. Human Immunomodulators

Human immunomodulators suitable for use as adjuvants in the invention include cytokines, such as interleukins e.g. IL-I, IL-2, IL-4, IL-5, IL-6, IL-7, IL-12 etc.) interferons, macrophage colony stimulating factor, and tumor necrosis factor.

### G. Bioadhesives and Mucoadhesives

Bioadhesives and mucoadhesives may also be used as adjuvants in the invention.

Suitable bioadhesives include esterified hyaluronic acid microspheres or mucoadhesives such as cross-linked derivatives of poly(acrylic acid), polyvinyl alcohol, polyvinyl pyrollidone, polysaccharides and carboxymethylcellulose. Chitosan and derivatives thereof may also be used as adjuvants in the invention.

### H. Microparticles

Microparticles may also be used as adjuvants in the invention.

Microparticles (i.e. a particle of 100 nm to 150 μm in diameter, more preferably 200 nm to 30 [mu]m in diameter, and most preferably 500 nm to 10 μm in diameter) formed from materials that are biodegradable and non-toxic (e.g. a poly(a-hydroxy acid), a polyhydroxybutyric acid, a polyorthoester, a polyanhydride, a polycaprolactone, etc.), with poly(lactide-co-glycolide) are preferred, optionally treated to have a negatively-charged surface (e.g. with SDS) or a positively-charged surface (e.g. with a cationic detergent, such as CTAB).

### I. Liposomes

Examples of lipo some formulations suitable for use as adjuvants are described in U.S. Pat. No. 6,090,406, U.S. Pat. No. 5,916,588 and EP0626169.

### J. Polyoxyethylene Ether and Polyoxyethylene Ester Formulations

Adjuvants suitable for use in the invention include polyoxyethylene ethers and polyoxyethylene esters. Such formulations further include polyoxyethylene sorbitan ester surfactants in combination with an octoxynol as well as polyoxyethylene alkyl ethers or ester surfactants in combination with at least one additional non-ionic surfactant such as an octoxynol. Preferred polyoxyethylene ethers are selected from the following group: polyoxyethylene-9-lauryl ether (laureth 9), polyoxyethylene-9-steoryl ether, polyoxytheylene-8-steoryl ether, polyoxyethylene-4-lauryl ether, polyoxyethylene-35-lauryl ether, and polyoxyethylene-23-lauiyl ether.

### K. Polyphosphazene (PCPP) PCPP Formulations are Described, for Example, in Andrianov et al. (1998) Biomaterials 19:109-115.

### L. Muramyl Peptides

Examples of muramyl peptides suitable for use as adjuvants in the invention include N-acetyl-muramyl-L-threonyl-D-isoglutamine (thr-MDP), N-acetyl-normuramyl-L-alanyl-D-isoglutamine (nor-MDP), and N-acetylmuramyl-L-alanyl-D-isoglutaminyl-L-alanine-2-(r-2′-dipalmitoyl-5n-glycero-3-hydroxyphosphoryloxy)-ethylamine MTP-PE).

### M. Imidazoquinolone Compounds.

Examples of imidazoquinolone compounds suitable for use adjuvants in the invention include Imiquamod and its homologues (e,g. ‘Resiquimod 3M’), described further in Stanley (2002) Clin Exp Dermatol 27:571-577.

The invention may also comprise combinations of aspects of one or more of the adjuvants identified above.

In one embodiment, the adjuvant is an aluminium salt, e.g. aluminium hydroxide.

### Methods of Treatment

The invention also provides a method for raising an immune response in a mammal, comprising administering a primer composition and a booster composition of the invention to the mammal. The immune response is preferably protective and preferably involves antibodies. The method may raise a booster response in a subject that has already been primed against N meningitidis by administration of the primer composition.

Subcutaneous and intranasal prime/boost regimes for OMVs are disclosed in Bakke et al. (2001) Infect. Immun. 69:5010-5015. The present invention may be performed using similar prime-boost regimes, except that the primer comprises OMVs whereas the boost comprises a meningococcal protein antigen (e.g an OMP).

The mammal (e.g. the subject referred to in any of the methods defined herein) is preferably a human. Where the vaccine is for prophylactic use, the human is preferably a child (e.g. a toddler or infant) or a teenager; where the vaccine is for therapeutic use, the human is preferably an adult. A vaccine intended for children may also be administered to adults e.g. to assess safety, dosage, immunogenicity, etc.

The invention also provides OMV and meningococcal protein antigen (e.g. OMP) compositions and mixtures of the invention for use as a medicament. The medicament is preferably able to raise an immune response in a mammal (i.e. it is an immunogenic composition) as part of a prime-boost regime as described herein and is more preferably a vaccine.

The invention also provides the use of OMV and meningococcal protein antigen (e.g. OMP) compositions in the manufacture of a combined vaccine for raising an immune response in a mammal, wherein the OMV composition is administered as a primer and the meningococcal protein antigen (e.g. OMP) composition is administered as a booster.

These uses and methods are preferably for the prevention and/or treatment of a disease caused by N. meningitidis e.g. bacterial (or, more specifically, meningococcal) meningitis, or septicemia.

One way of checking efficacy of therapeutic treatment involves monitoring Neisserial infection after administration of the composition of the invention. One way of checking efficacy of prophylactic treatment involves monitoring immune responses against OMV antigens after administration of the composition. Immunogenicity of compositions of the invention can be determined by administering them to test subjects (e.g. children 12-16 months age, or animal models) and then determining standard parameters including serum bactericidal antibodies (SBA) and ELISA titres (GMT). These immune responses will generally be determined around 4 weeks after administration of the composition, and compared to values determined before administration of the composition.

A SBA increase of at least 4-fold or 8-fold is preferred. Where more than one dose of the composition is administered, more than one post-administration determination may be made. Preferred compositions of the invention can confer an antibody titre in a subject that is superior to the criterion for seroprotection for an acceptable percentage of human subjects. Antigens with an associated antibody titre above which a host is considered to be seroconverted against the antigen are well known, and such titres are published by organisations such as WHO. Preferably more than 80% of a statistically significant sample of subjects is seroconverted, more preferably more than 90%, still more preferably more than 93% and most preferably 96-100%.

Compositions of the invention will generally be administered directly to a subject. Direct delivery may be accomplished by parenteral injection (e.g. subcutaneously, intraperitoneally, intravenously, intramuscularly, or to the interstitial space of a tissue), or by rectal, oral, vaginal, topical, transdermal, intranasal, ocular, aural, pulmonary or other mucosal administration. Intramuscular administration to the thigh or the upper arm is preferred. Injection may be via a needle (e.g. a hypodermic needle), but needle-free injection may alternatively be used. A typical intramuscular dose is 0.5 ml.

Dosage treatment can be a single dose schedule or a multiple dose schedule. Multiple doses may be used in a primary immunisation schedule and/or in a booster immunisation schedule. The primary dose schedule is followed by a booster dose schedule.

Suitable timing between priming doses (e.g. between 4-16 weeks), and between priming and boosting, can be routinely determined. The OMV-based RIVM vaccine was tested using a 3- or 4-dose primary schedule, with vaccination at 0, 2 & 8 or 0, 1, 2 & 8 months. MeNZB™ is administered as three doses at six week intervals. Similar priming dose regimes may be used according to the present invention, provided that the primary dose schedule (using an OMV preparation) is followed by a booster dose schedule (using an OMP preparation).

As described above, the priming dose schedule of the present invention may involve administration of vesicles from more than one serosubtype of N. meningitidis, either separately or in admixture.

Thus in one embodiment the method involves administering a meningococcal OMV preparation to a subject as a primer dose, and subsequently administering a meningococcal protein antigen (e.g. OMP) preparation to the subject as a booster dose. In one embodiment, the primer dose is given at time zero, and the booster dose is given between 2 weeks and 12 months, e.g. between 4 to 12 weeks after the primer dose. In another embodiment, one or more additional primer doses (e.g. 1 or 2 additional primer doses) are given between 0 and 6 weeks after a first primer dose, and one or more booster doses (e.g. 1 to 3 booster doses) are given between 6 weeks and 12 months after the first primer dose. The subject may receive the same or a different quantity of the primer and/or booster composition at each of the doses.

The invention may be used to elicit systemic and/or mucosal immunity.

In general, compositions of the invention are able to induce serum bactericidal antibody responses after being administered to a subject. These responses are conveniently measured in mice and are a standard indicator of vaccine efficacy (see for example Pizza et al. (2000) Science 287:1816-1820).

Serum bactericidal activity (SBA) measures bacterial killing mediated by complement, and can be assayed using human or baby rabbit complement. WHO standards relating to polysaccharide-based vaccines (but not specifically OMV- or protein-based vaccines) require a vaccine to induce at least a 4-fold rise in SBA in more than 90% of recipients. MeNZB™ elicits a 4-fold rise in SBA 4-6 weeks after administration of the third dose. According to the present invention, at least a 4-fold rise in SBA in more than 90% of recipients is preferably induced 4-6 weeks after administration of the booster composition.

The combined vaccines (i.e. primer plus booster compositions) of the present invention may additionally induce bactericidal antibody responses against one or more of hypervirulent lineages and against other lineages e.g. hyperinvasive lineages. Preferred groups of strains will include strains isolated in at least four of the following countries: GB, AU, CA, NO, IT, US, NZ, NL, BR, and CU.

The serum preferably has a bactericidal titre of at least 256 or higher, more preferably 512, 1024, 2048, 4096 or higher, e.g. the serum is able to kill at least 50% of test bacteria of a particular strain when diluted 1/1024, as described in Pizza et al. (2000) Science 287:1816-1820.

Preferred compositions can induce bactericidal responses against various meningococcal strains, including for example serogroup B meningococcus strains from cluster A4 (e.g. strain 961-5945 (B:2b:P1.21,16) and/or strain G2136), from ET-5 complex (e.g. strain MC58 (B:15:P1.7,16b) and/or strain 44/76 (B:15:P1.7,16)) or from lineage 3 (e.g. strain 394/98 (B:4:P1.4) and/or strain BZ198). Strains 961-5945 and G2136 are both Neisseria MLST reference strains (see Tettelin et al. (2000) Science 287:1809-1815, Pettersson et al. (1994) Microb Pathog 17(6):395-408 and http://neisseria.org/nm/typing/mlst/). In one embodiment the meningococcal strain is NZ98/254.

### Further Antigenic Components

As well as containing OMVs, in some embodiments primer compositions of the invention may include further non-vesicular antigens, such as a saccharide antigen from N. meningitidis, e.g. from serogroup A, C, W135 and/or Y. Where a saccharide or carbohydrate antigen is used, it is preferably conjugated to a carrier in order to enhance immunogenicity. Conjugation of meningococcal saccharide antigens is well known. The primer compositions may also comprise one or more antigens from a different pathogen, e.g. tetanus of diphtheria antigens. Preferably the primer composition does not comprise a purified meningococcal protein antigen.

Antigens in the composition will typically be present at a concentration of at least 1 μg/ml each. In general, the concentration of any given antigen will be sufficient to elicit an immune response against that antigen.

As an alternative to using protein antigens in the composition of the invention, nucleic acid encoding the antigen may be used. Protein components of the compositions of the invention may thus be replaced by nucleic acid (preferably DNA e.g. in the form of a plasmid) that encodes the protein.

As used herein, the term ‘comprising’ encompasses ‘including’ as well as ‘consisting’ e.g. a composition ‘comprising’ X may consist essentially or exclusively of X or may include an additional component.

It will be understood that the invention has been described by way of example only and modifications may be made whilst remaining within the scope and spirit of the invention.

### EXAMPLE

### Prime Boost Strategy to Optimize the Immune Response to Meningococcal Outer Membrane Antigens

In clinical trials immunization with outer membrane vesicles (OMVs) has been shown to offer protection against disease caused by Neisseria meningitidis (Bjune et al., 1991; Sierra et al., 1991). The OMVs are extracted from the organism in the presence of detergent and contain the same complement of protein antigens as the outer membrane of the intact bacterium (Poolman et al., 2006). The protection offered by OMV vaccines tends to be strain-specific, especially in the young who are most vulnerable to infection, because the predominant bactericidal antibody response is directed against the variable PorA antigen (Tappero et al., 1999). Vaccine developers have taken diametrically opposed approaches to this problem, either searching the genome for conserved protein antigens (Rappuoli, 2001) or developing formulations based on combinations of variable protein antigens such as PorA (van den Dobbelsteen et al., 2006; Urwin et al., 2004).

This example demonstrates a novel immunisation strategy to optimize the immune response to protective epitopes expressed on the meningococcal surface. In the present example, PorA is used as the outer membrane protein, but in alternative embodiments other conserved or other variable protein antigens may be used.

### Methods

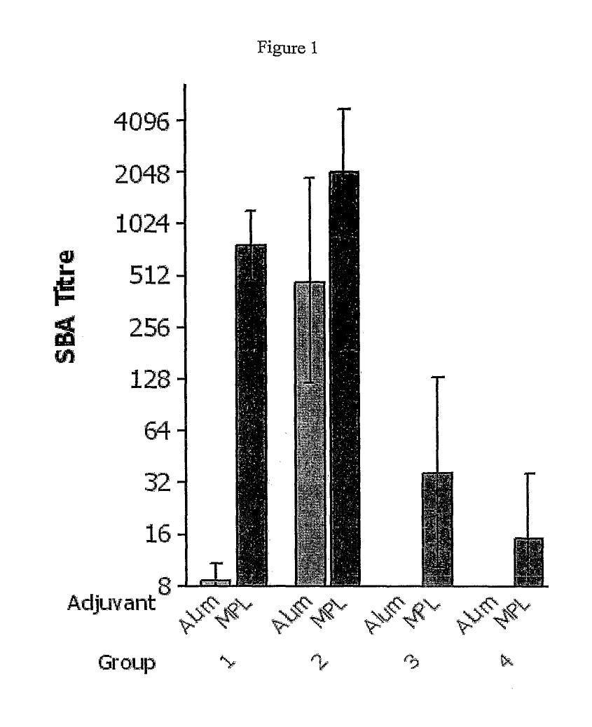

Female NIH mice (10/group) were immunised subcutaneously with either 1 μg of OMV (strain NZ98/254) or 10 μg or purified PorA protein (P1.7-2,4) priming and boosting as shown in FIG. 1. Booster doses were administered 3 weeks after priming. Either Aluminium Hydroxide (Alum) or MPL adjuvant (Monophosphoryl Lipid A+TDM, Sigma) was used. The mice were bled 2 weeks after the booster dose.

Vaccine protection induced in the mouse model to the PorA protein was determined by Serum Bactericidal Assays (SBAs) using baby rabbit complement and N. meningitidis strain Z6426/9140. The SBA titre was reported as the geometric mean of the reciprocal serum dilution yielding≧50% bacterial killing as compared with a viable count control.

Data was analysed by General Linear Models for analysis of covariance using Minitab 15.

### Results and Discussion

The results are shown in FIG. 1.

In this study the bactericidal responses to the PorA antigen after priming and boosting with various combinations of OMV and purified PorA protein components were compared. The results demonstrate that the presentation of the antigen used to prime the immune system (i.e. either as purified protein or in OMVs) had a larger effect on the bactericidal response than the presentation of the antigen used as the booster. Regardless of the adjuvant used, bactericidal antibody titres were highest in groups of mice primed with the OMV vaccine and boosted with purified PorA protein, demonstrating that it is possible to improve the bactericidal response elicited by OMVs by specifically targeting PorA (or other candidate antigen) with the booster dose.

Without being bound by theory, a possible explanation for these results is that priming with OMVs presents the protective PorA epitopes in the correct conformation for a good bactericidal response. Subsequent boosting with purified protein ensures that the response specifically targets the chosen antigen, in this case PorA. In addition, priming the immune response with OMVs ensures a strong Th1 response, in which subclasses of IgG that are most effective at complement mediated bactericidal killing predominate. This is consistent with the enhanced bactericidal response observed with the use of MPL, an adjuvant known to elicit a good Th1 type response.

Although PorA is immunodominant in OMV preparations, a number of other outer membrane protein antigens are currently under evaluation by vaccine developers including PorB, FetA, transferrin binding proteins, lactoferrin binding proteins, haem and haptoglobin receptors, complement factor H binding protein, NadA, Opa proteins, NspA, Omp85, PilQ and many other proteins of unconfirmed function that have been identified from genome sequence data. The principle of priming with an OMV and boosting with purified protein is equally applicable to these other antigens. For instance, in an alternative embodiment, the prime-boost strategy described here for PorA may be performed using the FetA antigen.

In contrast to their response to protein antigens, infants tend to respond poorly to immunization with OMVs. In embodiments of the present invention, use of a prime-boost strategy combining priming with OMVs and boost with purified outer membrane proteins may ensure a potent protective response in the very young, who are most at risk of meningococcal disease.

### References

Bjune,G., Hoiby, E. A., Gronnesby, J. K., Arnesen, O., Fredriksen, J. H., Halstensen, A. et al. (1991) Effect of outer membrane vesicle vaccine against group B meningococcal disease in Norway. Lancet 338: 1093-1096.

Poolman, J. T., Denoël, P., Feron, C., Goraj, K., and Weynants, V. (2006) Outer membrane Vesicle-based Meningococcal Vaccines. In Handbook of Meningococcal Disease. Frosch, M., and Maiden, M. (eds). WILEY-VCH, pp. 371-390.

Rappuoli,R. (2001) Reverse vaccinology, a genome-based approach to vaccine development. Vaccine 19: 2688-2691.

Sierra, V. G. G., Campa, C. H., Garcia, L. I., Sotolongo, F. P., Izquierdo, L. P., Valcarcel, M. N. et al. (1991) Efficacy evaluation of the Cuban vaccine VA-MENGOC-BC against disease caused by serogroup B Neisseria meningitidis. In Niesseria 1990. Achtman, M .e. a. (ed). Berlin: Walter de Gruyter, pp. 129-134.

Tappero, J. W., Lagos, R., Ballesteros, A. M., Plikaytis, B., Williams, D., Dykes, J. et al. (1999) Immunogenicity of 2 serogroup B outer-membrane protein meningococcal vaccines: a randomized controlled trial in Chile. JAMA 281: 1520-1527.

Urwin, R., Russell, J. E., Thompson, E. A., Holmes, E. C., Feavers, I. M., and Maiden, M. C. (2004) Distribution of Surface Protein Variants among Hyperinvasive Meningococci: Implications for Vaccine Design. Infect Immun 72: 5955-5962.

van den Dobbelsteen, G. P., van Dijken, H. H., Pillai, S., and van, A. L. (2006) Immunogenicity of a combination vaccine containing pneumococcal conjugates and meningococcal PorA OMVs. Vaccine.

Each of the applications and patents mentioned in this document, and each document cited or referenced in each of the above applications and patents, and any manufacturer's instructions or catalogues for any products cited or mentioned in each of the applications and patents and in any of the application cited documents, are hereby incorporated herein by reference. Furthermore, all documents cited in this text, and all documents cited or referenced in documents cited in this text, and any manufacturer's instructions or catalogues for any products cited or mentioned in this text, are hereby incorporated herein by reference.

Various modifications and variations of the described methods and system of the invention will be apparent to those skilled in the art without departing from the scope and spirit of the invention. Although the invention has been described in connection with specific preferred embodiments, it should be understood that the invention as claimed should not be unduly limited to such specific embodiments and that many modifications and additions thereto may be made within the scope of the invention. Indeed, various modifications of the described modes for carrying out the invention which are obvious to those skilled in immunology, molecular biology or related fields are intended to be within the scope of the claims. Furthermore, various combinations of the features of the following dependent claims can be made with the features of the independent claims without departing from the scope of the present invention.

1. A method of immunizing a subject against a disease caused by Neisseria meningitidis, comprising administering to the subject a primer composition comprising a meningococcal outer membrane vesicle preparation, and a booster composition comprising a meningococcal protein antigen preparation.

2. A method according to claim 1, wherein the disease is meningitis, septicaemia, meningococcal arthritis or pneumonia.

3. A method according to claim 1, wherein the disease is caused by Neisseria meningitidis serotype A, B, C, Y or W135.

4. A method according to claim 1, wherein the outer membrane vesicle preparation and/or the meningococcal protein antigen preparation is derived from Neisseria meningitidis serotype B.

5. A method according to claim 1, wherein the meningococcal protein antigen preparation is derived from Neisseria meningitidis strain NZ981254 or H44/76.

6. A method according to claim 1, wherein the primer composition comprises a RIVM, VA-MENGOC-BC MeNZB or MenBvac vaccine composition.

7. A method according to claim 1, wherein the meningococcal protein antigen preparation comprises a meningococcal outer membrane protein preparation.

8. A method according to claim 7, wherein the outer membrane protein preparation comprises one or more purified proteins selected from the group consisting of: PorA, PorB, FetA, NadA, transferrin binding proteins, lactoferrin binding proteins, haem and haptoglobin receptors, complement factor H binding protein, Opa protein, NspA, Omp85, PilQ, Cu, Zn-superoxide dismutase and immunogenic fragments thereof.

9. A method according to claim 8, wherein the outer membrane protein preparation comprises purified PorA or an immunogenic fragment thereof.

10. A method according to claim 1, wherein the outer membrane protein preparation is derived from Neisseria meningitidis serotype P1.7-2.4.

11. A method according to claim 1, wherein the primer composition and/or the booster composition further comprises an adjuvant.

12. A method according to claim 11, wherein the adjuvant comprises monophosphoryl lipid A or aluminium hydroxide.

13. A method according to claim 1, wherein the booster composition is administered 2 weeks to 12 months after the primer composition is administered to the subject.

14. A method of promoting an immune response in a subject against a meningococcus bacterium, comprising administering to the subject a primer composition comprising a meningococcal outer membrane vesicle preparation, and a booster composition comprising a meningococcal outer membrane protein preparation.

15. A pharmaceutical combination comprising a primer composition comprising a meningococcal outer membrane vesicle preparation, and a separate booster composition comprising a meningococcal protein antigen preparation.

16. (canceled)

17. (canceled)
